(19) United States
(12) Patent Application Publication
(10) Pub. No.: US 2004/0010142 A1
(43) Pub. Date: Jan. 15, 2004

(54) Novel process

(76) Inventors: Flemming Elmelund Nielsen, Virum (DK); Hanne T. Korno, Lyngby (DK); Kaare G. Rasmussen, Kobenhavn O (DK)

Correspondence Address: Reza Green, Esq., Novo Nordisk Pharmaceuticals, Inc., 100 College Road West, Princeton, NJ, 08540, US

(21) Appl. No.: 10/464,599

(22) Filed: Jun. 17, 2003

Related U.S. Application Data
(63) Continuation of application No. PCT/DK01/00836, filed on Dec. 19, 2001.
(60) Provisional application No. 60/257,385, filed on Dec. 22, 2000.

(30) Foreign Application Priority Data
Dec. 21, 2000 (DK) PA 2000 01920

(51) Int. Cl. C07D 285/22

(52) U.S. Cl. 544/12

(57) ABSTRACT

The present invention relates to a novel processes for preparing pharmaceutically active compounds of formula (I):

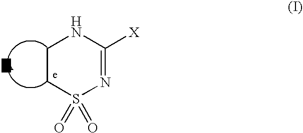

wherein A and X are defined in the description.

### CROSS REFERENCE TO RELATED APPLICATIONS

This application is a continuation of international application no. PCT/DKO1/00836 filed on Dec. 19, 2001, published in English, and claims the benefit of Danish application no. PA 2000 01920 filed on Dec. 21, 2000, and U.S. provisional application No. 60/257,385 filed on Dec. 22, 2000.

### FIELD OF THE INVENTION

The present invention relates to novel processes for preparing pharmaceutically active compounds and intermediates therefore.

Pharmaceutical active compounds acting as potent and selective potassium channel openers that, by inhibiting insulin release and inducing β-cell rest, can be used in treatment of Type I and Type II diabetes are described in WO 97/26265, WO 99/03861 and WO 00/37474.

Ways of synthesizing those compounds are described in WO 97/26265, pages 20 to 25, in WO 99/03861, pages 22 to 26 and in WO 00/37474, pages 12-16.

The present invention provides alternative methods of synthesis for the above mentioned compounds in a more efficient way.

### BACKGROUND OF THE INVENTION

In Tetrahedron Lett. 1998, 39, 2933; Tetrahedron Lett. 1998, 39, 2941 and Tetrahedron Lett. 1998, 39, 7979, the intermolecular coupling of phenyl derived boronic acids with amines, anilines, amides, imides, ureas, sulfonamides, carbamates and imidazoles is described.

It has now been found that boronic acid substituted 5 membered heterocycles can undergo an intramolecular coupling reaction with a guanidine moiety.

### DESCRIPTION OF THE INVENTION

The present invention provides novel processes for the preparation of fused 1,2,4-thiadiazine derivatives of the general formula (I):

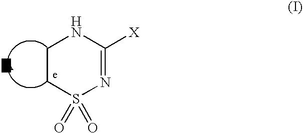

X is NR²R³, SR¹, S(═O)R¹, S(═O)₂R¹, or OR¹;

R¹ is hydrogen, C₃₋₆-cycloalkyl or (C₃₋₆-cycloalkyl)C₁₋₆-alkyl the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms, optionally being mono- or polysubstituted with halogen, cyano, trifluoromethyl, C₁₋₆-alkyl, C₁₋₆-alkoxy, C₁₋₆-alkoxy-C₁₋₆-alkyl, aryl, arylalkyl, hydroxy, oxo, nitro, amino, C₁₋₆-monoalkyl or dialkylamino; or straight or branched C₁₋₁₈-alkyl, C₂₋₁₈-alkenyl or C₂₋₁₈-alkynyl, each of the groups being optionally mono- or polysubstituted with halogen, hydroxy, C₁₋₆-alkoxy, C₁₋₆-alkylthio, C₃₋₆-cycloalkyl, nitro, amino, C₁₋₆- monoalkyl- or dialkylamino, cyano, oxo, formyl, acyl, carboxy, C₁₋₆-alkoxycarbonyl, carbamoyl, formylamino, C₁₋₆-alkylcarbonylamino, aryl, aryloxy, arylalkoxy; bicycloalkyl, aryl, heteroaryl, arylalkyl or heteroarylalkyl, each of the groups being optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;

R² is hydrogen; hydroxy; C₁₋₆-alkoxy; or C₁₋₆-alkyl, C₃₋₆-cycloalkyl, C₂₋₆- alkenyl or C₂₋₆-alkynyl optionally mono- or polysubstituted with halogen;

R³ is hydrogen, C₃₋₆-cycloalkyl or (C₃₋₆-cycloalkyl)C₁₋₆-alkyl, the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl; or

R³ is —OR⁴; —C(═Z)R⁴; —NR⁴R⁵; bicycloalkyl, aryl, heteroaryl, arylalkyl or heteroarylalkyl optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxy-carbonyl;

R⁴ is hydrogen; C₃₋₆-cycloalkyl or (C₃₋₆-cycloalkyl)C₁₋₆-alkyl, the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;

Z is O or S;

R⁵ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or

when R³ is —NR⁴R⁵, R⁴ and R⁵ together with the nitrogen atom form a 3-12 membered mono- or bicyclic system, in which one or more of the carbon atoms may be exchanged with nitrogen, oxygen or sulfur, each of these ring systems optionally being mono- or poly-substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or

when X is NR²R³, R² and R³ together with the nitrogen atom may form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;

A together with the carbon atoms forming bond e of formula (I) represents a 5 membered heterocyclic system comprising one or more nitrogen-, oxygen- or sulfur atoms, the heterocyclic systems optionally being mono- or polysubstituted with halogen; C₁₋₁₈-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, aryl, arylalkyl, aryloxy, the aryl group optionally being mono- or polysubstituted with C₁₋₆-alkyl, perhalomethyl, halogen, hydroxy or C₁₋₆-alkoxy; C₁₋₆-alkoxycarbonyl; C₁₋₆-alkoxycarbonyl-C₁₋₆-alkyl; carbamyl; carbamylmethyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thiocarbamyl; thioureido; C₁₋₆-monoalkyl- or dialkylaminothiocarbonyl- amino; C₁₋₆-monoalkyl- or dialkylaminosulfonyl; carboxy; carboxy-C₁₋₆-alkyl; acyl; formyl; or a 5-6 membered nitrogen, oxygen or sulfur containing ring, optionally substituted with C₁₋₆-alkyl or phenyl, the phenyl group optionally being mono- or polysubstituted with C₁₋₆-alkyl, perhalomethyl, halogen, hydroxy or C₁₋₆-alkoxy; or

a salt thereof with a pharmaceutically acceptable acid or base, or an optical isomer thereof, or a tautomeric form thereof, or metabolites or prodrugs thereof, comprising

a) reacting a compound of formula (II)

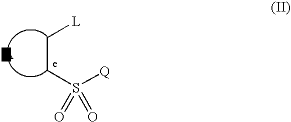

wherein A is as defined above, L is a leaving group selected from hydrogen, halogen or trimethylsilyl, and Q is halogen, with a compound of formula (III),

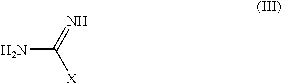

wherein X is NR²R³, wherein R² and R³ are defined above, or a suitable salt thereof, in the presence of a suitable base, to form a compound of formula (IV),

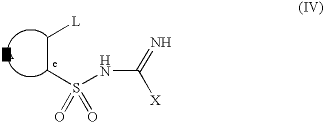

wherein A and L are as defined above and X is NR²R³; then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V),

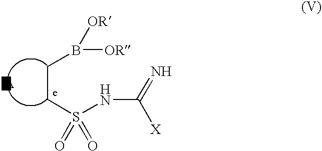

wherein A, R′ and R″ are as defined above and X is NR²R³, and thereupon cyclization of the compound of formula (V), optionally in the presence of a metal specie, to form the compound of formula (I); or

b) reacting a compound of formula (II)

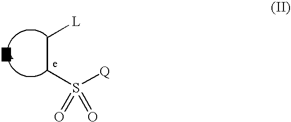

wherein A is as defined above, L is a leaving group selected from hydrogen, halogen or trimethylsilyl, and 0 is halogen, with a compound of formula (III),

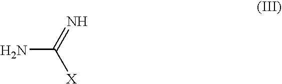

wherein X is SR¹, S(═O)R¹ or S(═O)₂R¹, wherein R¹ is defined above, or a suitable salt thereof, in the presence of a suitable base, to form a compound of formula (IV),

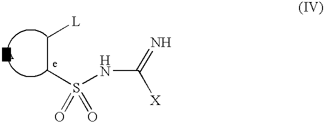

wherein A and L are as defined above and X is SR¹, S(═O)R¹ or S(═O)₂R¹, then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V),

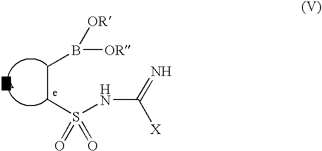

wherein A, R′ and R″ are as defined above and X is SR¹, S(═O)R¹ or S(═O)₂R¹, and thereupon cyclization of the compound of formula (V), optionally in the presence of a metal specie, to form the compound of formula (I); or

c) reacting a compound of formula (II)

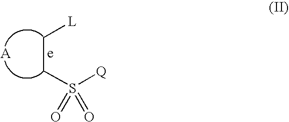

wherein A is as defined above, L is a leaving group selected from hydrogen, halogen or trimethylsilyl, and Q is halogen, with a compound of formula (III),

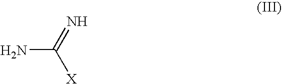

wherein X is OR¹, wherein R¹ is defined above, or a suitable salt thereof, in the presence of a suitable base, to form a compound of formula (IV)

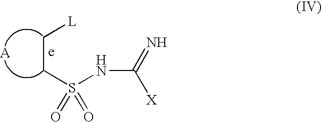

wherein A and L are as defined above and X is OR¹, then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V),

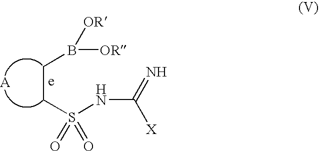

wherein A, R′ and R″ are as defined above and X is OR¹, and thereupon cyclization of the compound of formula (V), optionally in the presence of a metal specie, to form the compound of formula (I).

Within its scope the invention of the process for preparation of compounds of formula (I) includes all optical isomers of compounds of formula (I), some of which are optically active, and also their mixtures including racemic mixture thereof.

The scope of the invention also includes all tautomeric forms of the compounds of formula (I) as well as metabolites or prodrugs of a compound of formula (I).

The salts include pharmaceutically acceptable acid addition salts, pharmaceutically acceptable metal salts or optionally alkylated ammonium salts, such as hydrochloric, hydrobromic, hydroiodic, phosphoric, sulfuric, trifluoroacetic, trichloroacetic, oxalic, maleic, pyruvic, malonic, succinic, citric, tartaric, fumaric, mandelic, benzoic, cinnamic, methanesulfonic, ethanesulfonic, picric and the like, and include acids related to the pharmaceutically acceptable salts listed in Journal of Pharmaceutical Science, 66, 2 (1977) and incorporated herein by reference, or lithium, sodium, potassium, magnesium and the like.

A “metabolite” of a compound disclosed in this application is an active derivative of a compound disclosed herein which is produced when the compound is metabolized. Metabolites of compounds disclosed herein can be identified either by administration of a compound to a host and an analysis of blood samples from the host, or by incubation of compounds with hepatic cells in vitro and analysis of the incubant. A “prodrug” is a compound that either is converted into a compound disclosed in the application in vivo or has the same active metabolite as a compound disclosed in this application.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms e.g. methoxy, ethoxy, propoxy, isopropoxy, butoxy, pentoxy.

The terms “C₂₋₆-alkenyl” and “C₂₋₁₈-alkenyl” as used herein refers to an unsaturated hydrocarbon chain having 2-6 or 2-18 carbon atoms and one double bond such as e.g. vinyl, 1-propenyl, allyl, isopropenyl, n-butenyl, n-pentenyl and n-hexenyl.

The term “C₃₋₆-cycloalkyl” as used herein refers to a radical of a saturated cyclic hydrocarbon with the indicated number of carbons such as cyclopropyl, cyclobutyl, cyclopentyl, or cyclohexyl.

The terms “C₂₋₆-alkynyl” and “C₂₋₁₈-alkynyl” as used herein refers to unsaturated hydrocarbons which contain triple bonds, such as e.g. —C≡CH, —C≡CCH₃, —CH₂C≡CH, —CH₂CH₂C≡CH, —CH(CH₃)C≡CH, and the like.

The term “C₁₋₆-alkoxy-C₁₋₆-alkyl” as used herein refers to a group of 2-12 carbon atoms interrupted by an O such as e.g. CH₂—O—CH₃, CH₂—O—CH₂—CH₃, CH₂—O—CH(CH₃)₂ and the like.

The term “halogen” means fluorine, chlorine, bromine or iodine.

The term “perhalomethyl” means trifluoromethyl, trichloromethyl, tribromomethyl or triiodomethyl.

The terms “C₁₋₆-alkyl”, “C₁₋₈-alkyl”, “C₁₋₁₂-alkyl” and “C₁₋₁₈-alkyl” as used herein, alone or in combination, refers to a straight or branched, saturated hydrocarbon chain having the indicated number of carbon atoms such as e.g. methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-hexyl, 1,2-dimethylpropyl, 2,2-dimethylpropyl, 1,2,2-trimethylpropyl and the like. The term “C₁₋₁₈-alkyl” as used herein also includes secondary C3-6-alkyl and tertiary C₄₋₆-alkyl.

The term “C₁₋₆-monoalkylamino” as used herein refers to an amino group wherein one of the hydrogen atoms is substituted with a straight or branched, saturated hydrocarbon chain having the indicated number of carbon atoms such as e.g. methylamino, ethylamino, propylamino, n-butylamino, sec-butylamino, isobutylamino, tert-butylamino, n-pentylamino, 2-methylbutylamino, n-hexylamino, 4-methylpentylamino, neopentylamino, n-hexylamino, 2,2-dimethylpropylamino and the like.

The term “C₁₋₆-dialkylamino” as used herein refers to an amino group wherein the two hydrogen atoms independently are substituted with a straight or branched, saturated hydrocarbon chain having the indicated number of carbon atoms; such as dimethylamino, N-ethyl-N-methylamino, diethylamino, dipropylamino, N-(n-butyl)-N-methylamino, di(n-pentyl)-amino, and the like.

The term “acyl” as used herein refers to a monovalent substituent comprising a C₁₋₆-alkyl group linked through a carbonyl group; such as e.g. acetyl, propionyl, butyryl, isobutyryl, pivaloyl, valeryl, and the like.

The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, sec-butoxycarbonyl, tert-butoxycarbonyl, 3-methylbutoxycarbonyl, n-hexoxycarbonyl and the like.

The term “3-12 membered mono- or bicyclic system” as used herein refers to a monovalent substituent of formula —NR²R³ where R² and R³ together with the nitrogen atom form a 3-12 membered mono- or bicyclic system, in which one or more of the carbon atoms may be exchanged with nitrogen, oxygen or sulfur, such as 1-pyrrolidyl, piperidino, morpholino, thiomorpholino, 4-methylpiperazin-1-yl, 7-azabicyclo[2.2.1]heptan-7-yl, tropanyl and the like.

The term “4-12 membered mono- or bicyclic system” as used herein refers to a monovalent substituent of formula B(OR′)(OR″) where R′ and R″ together with the boron atom form a 4-12 membered mono- or bicyclic system, in which one or more of the carbon atoms may be exchanged with nitrogen, oxygen or sulfur, such as 1,3,2-dioxaborolan-2-yl, 4,4,5,5-tetramethyl-1,3,2-dioxaborolan-2-yl, 1,3,2-dioxaborinan-2-yl, 5,5-dimethyl-1,3,2-dioxaborinan-2-yl, 1,3,2-benzodioxaborol-2-yl, and the like.

The term “3-6 membered saturated ring system” as used herein refers to a monovalent substituent comprising a monocyclic saturated system containing one or more hetero atoms selected from nitrogen, oxygen and sulfur and having 3-6 members and having its free valence from a carbon atom, e.g. 2-pyrrolidyl, 4-piperidyl, 3-morpholinyl, 1,4-dioxan-2-yl, 5-oxazolidinyl, 4-isoxazolidinyl, or 2-thiomorpholinyl.

The term “bicycloalkyl” as used herein refers to a monovalent substituent comprising a bicyclic structure made of 6-12 carbon atoms such as e.g. 2-norbornyl, 7-norbornyl, 2-bicyclo[2.2.2]octyl, and 9-bicyclo[3.3.1]nonanyl.

The term “aryl” as used herein refers to phenyl, 1-naphthyl, or 2-naphthyl.

The term “heteroaryl” as used herein, alone or in combination, refers to a monovalent substituent comprising a 5-6 membered monocyclic aromatic system or a 9-10 membered bicyclic aromatic system containing one or more heteroatoms selected from nitrogen, oxygen and sulfur, e.g. pyrrole, imidazole, pyrazole, triazole, pyridine, pyrazine, pyrimidine, pyridazine, isothiazole, isoxazole, oxazole, oxadiazole, thiadiazole, quinoline, isoquinoline, quinazoline, quinoxaline, indole, benzimidazole, benzofuran, pteridine, and purine.

The term “arylalkyl” as used herein refers to a straight or branched saturated carbon chain containing from 1 to 6 carbons substituted with an aromatic carbohydride; such as benzyl, phenethyl, 3-phenylpropyl, 1-naphtylmethyl, 2-(1-naphtyl)ethyl and the like.

The term “aryloxy” as used herein refers to phenoxy, 1-naphthyloxy or 2-naphthyloxy.

The term “arylalkoxy” as used herein refers to a C₁₋₆-alkoxy group substituted with an aromatic carbohydride, such as benzyloxy, phenethoxy, 3-phenylpropoxy, 1-naphthylmethoxy, 2-(1-naphtyl)ethoxy and the like.

The term “C₁₋₆-alkylsulfonyl” as used herein refers to a monovalent substituent comprising a C₁₋₆-alkyl group linked through a sulfonyl group such as e.g. methylsulfonyl, ethylsulfonyl, n-propylsulfonyl, isopropylsulfonyl, n-butylsulfonyl, sec-butylsulfonyl, isobutylsulfonyl, tert-butylsulfonyl, n-pentylsulfonyl, 2-methylbutylsulfonyl, 3-methylbutylsulfonyl, n-hexylsulfonyl, 4-methylpentylsulfonyl, neopentylsulfonyl, n-hexylsulfonyl and 2,2-dimethylpropylsulfonyl.

The term “C₁₋₆-monoalkylaminosulfonyl” as used herein refers to a monovalent substituent comprising a C₁₋₆-monoalkylamino group linked through a sulfonyl group such as e.g. methylaminosulfonyl, ethylaminosulfonyl, n-propylaminosulfonyl, isopropylaminosulfonyl, n-butylaminosulfonyl, sec-butylaminosulfonyl, isobutylaminosulfonyl, tert-butylaminosulfonyl, n-pentylaminosulfonyl, 2-methylbutylaminosulfonyl, 3-methylbutylaminosulfonyl, n-hexylaminosulfonyl, 4-methylpentylaminosulfonyl, neopentylaminosulfonyl, n-hexylaminosulfonyl and 2,2-dimethylpropylaminosulfonyl.

The term “C₁₋₆-dialkylaminosulfonyl” as used herein refers to a monovalent substituent comprising a C₁₋₆-dialkylamino group linked through a sulfonyl group such as dimethylaminosulfonyl, N-ethyl-N-methylaminosulfonyl, diethylaminosulfonyl, dipropylaminosulfonyl, N-(n-butyl)-N-methylaminosulfonyl, di(n-pentyl)aminosulfonyl, and the like.

The term “C₁₋₆-alkylsulfinyl” as used herein refers to a monovalent substituent comprising a straight or branched C₁₋₆-alkyl group linked through a sulfinyl group (—S(═O)—); such as e.g. methylsulfinyl, ethylsulfinyl, isopropylsulfinyl, butylsulfinyl, pentylsulfinyl, and the like.

The term “C₁₋₆-alkylcarbonylamino” as used herein refers to an amino group wherein one of the hydrogen atoms is substituted with an acyl group, such as e.g. acetamido, propionamido, isopropylcarbonylamino, and the like.

The term “(C₃₋₆-cycloalkyl)C₁₋₆-alkyl” as used herein, alone or in combination, refers to a straight or branched, saturated hydrocarbon chain having 1 to 6 carbon atoms and being monosubstituted with a C₃₋₆-cycloalkyl group, the cycloalkyl group optionally being mono- or polysubstituted with C₁₋₆-alkyl, halogen, hydroxy or C₁₋₆-alkoxy; such as e.g. cyclopropylmethyl, (1-methylcyclopropyl)methyl, 1-(cyclopropyl)ethyl, cyclopentylmethyl, cyclohexylmethyl, and the like.

The term “C₁₋₆-alkylthio” or “C₁₋₆-alkylsulfanyl” as used herein, alone or in combination, refers to a straight or branched monovalent substituent comprising a lower alkyl group linked through a divalent sulfur atom having its free valence bond from the sulfur atom and having 1 to 6 carbon atoms e.g. methylsulfanyl, ethylsulfanyl, propylsulfanyl, butylsulfanyl, pentylsulfanyl.

The term “arylthio” or “arylsulfanyl” as used herein, alone or in combination, refers to an aryl group linked through a divalent sulfur atom having its free valence bond from the sulfur atom, the aryl group optionally being mono- or polysubstituted with C₁₋₆-alkyl, halogen, hydroxy or C₁₋₆-alkoxy; e.g. phenylsulfanyl, (4-methylfenyl)sulfanyl, (2-chlorophenyl)sulfanyl, and the like.

The term “arylsulfinyl” as used herein refers to an aryl group linked through a sulfinyl group (—S(═O)—), the aryl group optionally being mono- or polysubstituted with C₁₋₆-alkyl, halogen, hydroxy or C₁₋₆-alkoxy; such as e.g. phenylsulfinyl, (4-chlorophenyl)sulfinyl, and the like.

The term “arylsulfonyl” as used herein refers to an aryl group linked through a sulfonyl group, the aryl group optionally being mono- or polysubstituted with C₁₋₆-alkyl, halogen, hydroxy or C₁₋₆-alkoxy; such as e.g. phenylsulfonyl, tosyl, and the like.

The term “C₁₋₆-monoalkylaminocarbonyl” as used herein refers to a monovalent substituent comprising a C₁₋₆-monoalkylamino group linked through a carbonyl group such as e.g. methylaminocarbonyl, ethylaminocarbonyl, n-propylaminocarbonyl, isopropylaminocarbonyl, n-butylaminocarbonyl, sec-butylaminocarbonyl, isobutylaminocarbonyl, tert-butylaminocarbonyl, n-pentylaminocarbonyl, 2-methylbutylaminocarbonyl, 3-methylbutylamino-carbonyl, n-hexylaminocarbonyl, 4-methylpentylaminocarbonyl, neopentylaminocarbonyl, n-hexylaminocarbonyl and 2-2-dimethylpropylaminocarbonyl.

The term “C₁₋₆-dialkylaminocarbonyl” as used herein refers to a monovalent substituent comprising a C₁₋₆-dialkylamino group linked through a carbonyl group such as dimethylaminocarbonyl, N-ethyl-N-methylaminocarbonyl, diethylaminocarbonyl, dipropylaminocarbonyl, N-(n-butyl)-N-methylaminocarbonyl, di(n-pentyl)aminocarbonyl, and the like.

The term “C₁₋₆-monoalkylaminocarbonylamino” as used herein refers to an amino group wherein one of the hydrogen atoms is substituted with a C₁₋₆-monoalkylaminocarbonyl group, e.g. methylaminocarbonylamino, ethylaminocarbonylamino, n-propylaminocarbonylamino, isopropylaminocarbonylamino, n-butylaminocarbonylamino, sec-butylaminocarbonylamino, isobutylaminocarbonylamino, tert-butylaminocarbonylamino, and 2-methylbutylaminocarbonylamino.

The term “C₁₋₆-dialkylaminocarbonylamino” as used herein refers to an amino group wherein one of the hydrogen atoms is substituted with a C₁₋₆-dialkylaminocarbonyl group, such as dimethylaminocarbonylamino, N-ethyl-N-methylaminocarbonylamino, diethylaminocarbonylamino, dipropylaminocarbonylamino, N-(n-butyl)-N-methylaminocarbonylamino, di(n-pentyl)aminocarbonylamino, and the like.

The term “5-membered heterocyclic system” as used herein refers to: a monocyclic unsaturated or saturated system containing one, two or three hetero atoms selected from nitrogen, oxygen and sulfur and having 5 members, e.g. pyrrole, furan, thiophene, pyrroline, dihydrofuran, dihydrothiophene, imidazole, imidazoline, pyrazole, pyrazoline, oxazole, thiazole, isoxazole, isothiazole, 1,2,3-oxadiazole, furazan, 1,2,3-triazole, 1,2,3-thiadiazole or 2,1,3-thiadiazole.

The term “5- or 6-membered nitrogen, oxygen or sulfur containing ring” as used herein refers to a monovalent substituent comprising a monocyclic unsaturated or saturated system containing one or more nitrogen, oxygen or sulfur atoms and having 5 or 6 members, e.g. pyrrolidinyl, pyrrolinyl, imidazolidinyl, pyrazolidinyl, pyrazolinyl, piperidyl, piperazinyl, pyrrolyl, 2H-pyrrolyl, imidazolyl, pyrazolyl, triazolyl, pyridyl, pyrazinyl, pyrimidinyl, pyridazinyl, morpholino, thiomorpholino, isothiazolyl, isoxazolyl, oxazolyl, oxadiazolyl, thiadiazolyl, 1,3-dioxolanyl, and 1,4-dioxolanyl.

The term “base” as used herein refers to inorganic and organic bases, which can be used to make a certain transformation taking place. Useful bases are: C₁₋₁₈-alkyl lithium, aryl lithium, C₁₋₁₈-alkyl magnesium halogenides. Hydroxides as e.g. sodium, lithium, magnesium, calcium, barium, potassium or cesium hydroxide. Carbonates as e.g. sodium, lithium, magnesium, calcium, barium, potassium or cesium carbonate. Hydrogen carbonates as e.g. sodium, lithium, magnesium, calcium, barium, potassium or cesium hydrogen carbonate. Alcoholates of sodium, lithium, magnesium, calcium, barium, potassium or cesium. Alcoholates of t-butanol, methanol, ethanol, 1-propanol, 2-propanol. Tertiary amines as e.g. dimethylaminopyridine, triethylamine, diisopropylethylamine (DIPEA), pyridine, 1,5-diazabicyclo[4.3.0]non-5-ene (DBN), 1,4-diazabicyclo[2.2.2]octane (DABCO, TED), 1,8-diazabicyclo[5.4.0]undec-7-ene (DBU). Phosphates as e.g. sodium, lithium, magnesium, calcium, barium, potassium or cesium phosphate. Sulfates as e.g. sodium, lithium, magnesium, calcium, barium, potassium or cesium sulfate. Secondary amine bases as e.g. sodium, lithium, magnesium, calcium, barium, potassium or cesium bis(isopropyl) amide and bis(cyclohexyl)amides and e.g. sodium, magnesium, calcium, barium, potassium or cesium bis(trimetylsilyl)amide. Hydrides as e.g. sodium hydride and potassium hydride. Carboxylic acid salts as e.g. sodium, lithium, magnesium, calcium, barium, potassium or cesium formate, acetate, propionate.

The term “metal specie” as used herein refers to all metal compounds, which are capable of promoting the transformation taking place at lower temperatures or similar mild conditions; such as e.g. copper or a copper (I) or copper (II) salt.

In one embodiment of the invention X is NR²R³.

In another embodiment of the invention R² is hydrogen or C₁₋₆-alkyl.

In another embodiment of the invention R³ is hydrogen, C₃₋₆-cycloalkyl, (C₃₋₆-cycloalkyl)C₁₋₆-alkyl or straight or branched C₁₋₁₈-alkyl.

In another embodiment of the invention A together with the carbon atoms forming bond e of formula (I) represents a 5 membered heterocyclic system comprising one sulfur atom, the heterocyclic system optionally being substituted with halogen.

In another embodiment of the invention selected compounds of formula (I) are:

3-Amino-6-chloro-4H-thieno[3,2-e]-1,2,4-thiadiazine 1,1-dioxide,

6-Chloro-3-isopropylamino-4H-thieno[3,2-e]-1,2,4-thiadiazine 1,1-dioxide,

6-Chloro-3-(1-methylcyclopropyl)amino-4H-thieno[3,2-e]-1,2,4-thiadiazine 1,1-dioxide,

6-Chloro-3-ethylamino-4H-thieno[3,2-e]-1,2,4-thiadiazine 1,1-dioxide,

6-Chloro-3-octylamino-4H-thieno[3,2-e]-1,2,4-thiadiazine 1,1-dioxide, or

a salt thereof with a pharmaceutically acceptable acid or base, or an optical isomer thereof, or a tautomeric form thereof, or metabolites or prodrugs thereof.

In one embodiment of the invention R′ and R″ are hydrogen.

In one embodiment of the invention OR′ and OR″ together with the boron atom form a 1,3,2-dioxaborolan-2-yl, 4,4,5,5-tetramethyl-1,3,2-dioxaborolan-2-yl, 1,3,2-dioxaborinan-2-yl, 5,5-dimethyl-1,3,2-dioxaborinan-2-yl or a 1,3,2-benzodioxaborol-2-yl group.

In one embodiment of the invention the bases are selected from the following organic and inorganic bases: C₁₋₁₈-alkyl lithium, aryl lithium, C₁₋₁₈-alkyl magnesium halides, triethylamine, pyridine, hydroxides, carbonates or hydrogen carbonates of sodium, lithium, magnesium, calcium, barium, potassium or cesium.

In another embodiment of the invention the bases are selected from sodium hydroxide, potassium carbonate, cesium carbonate, potassium hydroxide, pyridine, triethylamine, butyl lithium, hexyl lithium, isopropyl magnesium chloride.

In one embodiment of the invention the “metal specie” is selected from: copper bronze, copper oxide, copper chloride, copper bromide or copper iodide, copper fluoride, copper acetate, copper acetylacetonate, copper butyrate, copper carbonate, copper cyclohexanebutyrate, copper diiron tetraoxide, copper gluconate, copper formate, copper hexaflouroacetylacetonate, copper methoxide, copper naphtenate, copper oxalate, copper perchlorate, copper phenylacetylide, copper phthalocyanide, copper selenide, copper sulfate, copper sulfide, copper tartrate, copper tetrafluoroborate, copper thiocyanate, copper triflouroacetylacetonate, copper triflouromethansulfonate, copper tungstate.

In another embodiment of the invention the “metal specie” is selected from copper bronze, copper oxide, copper chloride, copper bromide, copper iodide, copper acetate.

The present invention is illustrated by the following examples.

The starting materials are either known compounds or compounds, which may be prepared in analogy with the preparation of known compounds or in analogy with known methods.

### EXAMPLE 1

3-Amino-6-chloro-4H-thieno[3,2-e]-1,2,4-thiadiazine 1,1-dioxide

a) N-(5-Chloro-3-(dihydroxyboryl)-2-thienylsulfonyl)quanidine

N-(3-Bromo-5-chloro-2-thienylsulfonyl)guanidine (2.0 g, 1.69 mmol) in tetrahydrofuran (30 ml) was cooled to below −60° C. and n-butyllithium (24 ml of a 1.6M solution in hexane, 38 mmol) was added at such a rate that the temperature did not exceed −59° C. The mixture was stirred at this temperature for 1.5 h. Then trimethyl borate (5.2 ml, 46 mmol) was added, and the reaction was stirred for 2 h at −60° C. The mixture was allowed to reach room temperature. Ethyl acetate (40 ml) and water (50 ml) was added, and the mixture was acidified to pH 2-3 with 3M hydrochloric acid. The phases were separated, and the organic phase was washed with water. The combined organic extracts were dried over sodium sulfate and the solvent was removed under reduced pressure giving 0.34 g (72%) of the title compound as a solid product: LC-MS: m/z 284/286 (M+1)⁺.

b) 3-Amino-6-chloro-4H-thieno[3,2-e]-1,2,4-thiadiazine 1,1-dioxide

A mixture of N-(5-chloro-3-(dihydroxyboryl)-2-thienylsulfonyl)guanidine (0.2 g, 0.7 mmol), pyridine (0.17 ml, 2.1 mmol), and copper(II) acetate (0.16 g, 0.88 mmol) in 1-methyl-2-pyrrolidinone (10 ml) was stirred at room temperature for 2 h. The mixture was filtered and the filtrate was treated with water (40 ml), ethyl acetate (30 ml) and 3M hydrochloric acid to pH 3. The phases were separated and the organic phase was washed with brine, dried over sodium sulfate, and concentrated under reduced pressure giving the title compound (0.22 g, 44%): mp>361° C.; ¹H-NMR (DMSO-d₆): δ 7.02 (s, 1H), 7.10 (br s, 2H), 11.18 (s, 1H).

### EXAMPLE 2

6-Chloro-3-isopropylamino-4H-thieno[3,2-e]-1,2,4-thiadiazine 1,1-dioxide

a) N-(5-Chloro-3-(dihydroxyboryl)-2-thienylsulfonyl)-N′-isopropylquanidine

n-Butyllithium (43 ml, 69 mmol) was added to a solution of N-(3-bromo-5-chloro-2-thienylsulfonyl)-N′-isopropylguanidine (5.0 g, 13.9 mmol) in tetrahydrofuran (50 ml) under a nitrogen atmosphere at −60° C. After stirring at this temperature for 2 h trimethyl borate (9.5 ml, 80.7 mmol) was added. The mixture was stirred at −60° C. for 3 h, and then it was allowed to reach room temperature, ethyl acetate (50 ml) and water (50 ml) was added, the mixture was acidified to pH 3 with 3M hydrochloric acid, and stirred for 3 h at room temperature. The phases were separated and the organic phase was dried with sodium sulfate. The solvent was removed under reduced pressure giving 3.6 g (80%) of the title compound as a thick oil: LC-MS: m/z 326/327 (M+1)⁺.

b) 6-Chloro-3-isopropylamino-4H-thieno[3,2-e]-1,2,4-thiadiazine 1,1-dioxide

A mixture of N-(5-chloro-3-(dihydroxyboryi)-2-thienylsulfonyl)-N′-isopropylguanidine (1 g, 3.1 mmol), copper(II) acetate (0.56 g, 3.1 mmol) and pyridine (0.75 ml, 9.3 mmol) was stirred in 1-methyl-2-pyrrolidinone (40 ml) for 2 h. The mixture was filtered and the filter cake washed with 1-methyl-2-pyrrolidinone. Water (80 ml) and ethyl acetate (80 ml) was added and the mixture was acidified with 3M hydrochloric acid to pH 6. The phases were separated and the organic phase was dried over sodium sulfate. The solvent was removed under reduced pressure giving 0.65 g (75%) of the title compound: mp 281-283° C.; ¹H-NMR (DMSO-d₆): δ 1.15 (d, 6H), 3.88 (m, 1H), 7.08 (s, 1H), 7.20 (br d, 1H), 10.74 (br s, 1H); MS m/z: 279/281 (M⁺)

### EXAMPLE3

6-Chloro-3-(1-methylcyclopropyl)amino-4H-thieno[3,2-e]-1,2,4-thiadiazine 1,1-dioxide

a) N-(5-Chloro-3-(dihydroxyboryl)-2-thienylsulfonyl)-N′-(1-methylcyclopropyl)quanidine

Procedure A

N-(3-Bromo-5-chloro-2-thienylsulfonyl)-N′-(1-methylcyclopropyl)guanidine (5.0 g, 13.4 mmol) in tetrahydrofuran (50 ml) was cooled to −60° C. and n-butyllithium (48 ml of a 1.6 M solution in hexane , 76 mmol) was added at such a rate that the temperature was kept at −60° C. The mixture was stirred for 1.5 h, then trimethyl borate (9.4 ml, 82.8 mmol) was added, and the reaction was stirred at −60° C. for 3 h. The mixture was allowed to reach room temperature and ethyl acetate (40 ml) and water (40 ml) was added followed by acidification to pH 3 with 3M hydrochloric acid. The phases were separated and the aqueous phase was extracted with ethyl acetate (2×20 ml). The combined organic phases were dried over sodium sulfate and the solvent was removed under reduced pressure giving 3.3 g (74%) of the title compound as a solid product.

Procedure B

i) N-(5-Chloro-2-thienylsulfonyl)-N′-(1-methylcyclopropyl)quanidine

A solution of 5-chloro-2-thiophenesulfonyl chloride (2.17 g, 10 mmol) in ethyl ether (20 ml) was added to a stirred solution of N-(1-methylcyclopropyl)guanidine hydrochloride (1.48 g, 10 mmol) in 1 N sodium hydroxide (20 ml) during 5 min, and the mixture was stirred at room temperature for 1 h. The resulting precipitate was isolated by filtration, washed with ethyl ether and dried to give 2.44 g (83%) of the title compound as a white solid: ¹H-NMR (DMSO-d₆): δ 0.65 (br s, 4H), 1.25 (s, 3H), 7.12 (d, 1H), 7.34 (d, 1H), 6.5-8.0 (broad peaks, 3H); LC-MS: m/z 294/296 (M+1)⁺.

ii) N-(5-Chloro-3-(dihydroxyboryl)-2-thienylsulfonyl)-N′-(1-methylcyclopropyl)guanidine

To a stirred solution of N-(5-chloro-2-thienylsulfonyl)-N′-(1-methylcyclopropyl)guanidine (1.0 g, 3.4 mmol) in dry tetrahydrofuran (40 ml) was added n-butyllithium (5.4 ml of a 2.5 M solution in hexane, 13.5 mmol) at −40° C. under nitrogen. After stirring at −20° C. for 30 min, the mixture was cooled to −70° C. and trimethyl borate (2.8 ml, 25 mmol) was added. The mixture was stirred at −70° C. for an additional hour and then allowed to reach room temperature, acidified with 0.2 M hydrochloric acid, and extracted with ethyl acetate (2×100 ml). The organic phase was washed with water, dried over sodium sulfate and evaporated to give 1.17 g of the crude title compound as a pale yellow oil. Purification by flash chromatography (dichloromethane/methanol (19:1) afforded 203 mg (18%) of the pure product as a white solid: ¹H-NMR (DMSO-d₆): δ 0.65 (br s, 4H), 1.23 (s, 3H), 7.13 (s, 1H), 6.5-8.0 (broad peaks, 3H), 8.39 (s, 2H); LC-MS: m/z 339/341 (M+1)⁺.

b) 6-Chloro-3-(1-methylcyclopropyl)amino-4H-thieno[3,2-e]-1,2,4-thiadiazine 1,1-dioxide

A mixture of N-(5-chloro-3-(dihydroxyboryl)-2-thienylsulfonyl)-N′-(1-methylcyclopropyl)-guanidine (0.5 g, 1.0 mmol), pyridine (0.24 ml, 3.0 mmol), 4A molecular sieves (0.7 g) and copper(II) acetate (0.18 g, 1.0 mmol) in 1-methyl-2-pyrrolidinone (20 ml) was stirred at room temperature for 3 h. The mixture was filtered and the filtrate was treated with water (125 ml) and 1M oxalic acid to pH<6. The precipitate was removed by filtration and the product was recovered from the filtrate by extraction with ethyl acetate (3×30 ml). The combined organic phases were washed with brine, dried over sodium sulfate, and concentrated under reduced pressure giving the title compound (0.12 g, 40%): mp 258° C.; ¹H-NMR (DMSO-d₆): δ 0.67 (m, 4H), 1.33 (s, 3H), 7.11 (br s, 1H), 7.89 (br s, 1H), 11.25 (br s, 1H).

### EXAMPLE 4

6-Chloro-3-(1-methylcyclopropyl)amino-4H-thieno[3,2-e]-1,2,4-thiadiazine 1,1-dioxide

a) N-(5-Chloro-3-(5,5-dimethyl-1,3,2-dioxaborinan-2-yl)-2-thienylsulfonyl)-N′-(1-methylcyclopropyl)quanidine

N-(5-Chloro-3-(dihydroxyboryl)-2-thienylsulfonyl)-N′-(1-methylcyclopropyl)guanidine was dissolved in acetonitrile (6 ml) with stirring under nitrogen and 2,2-dimethyl-1,3-propandiol (0.19 g, 1.8 mmol) was added. The mixture was stirred at room temperature overnight. The precipitated solid was isolated by filtration giving 0.37 g (83%) of the title compound: ¹H-NMR (DMSO-d₆): δ 0.65 (m, 4H), 1.00 (s, 6H), 1.33 (s, 3H), 3.70 (s, 4H), 7.06 (br s, 1H), 7.70 (br s, 1H), 8.40 (br s, 1H).

b) 6-Chloro-3-(1-methylcyclopronyl)amino-4H-thieno[3,2-e]-1,2,4-thiadiazine 1,1-dioxide

N-(5-Chloro-3-(5,5-dimethyl-1,3,2-dioxaborinan-2-yl)-2-thienylsulfonyl)-N′-(1-methylcyclopropyl)guanidine (0.2 g, 0.5 mmol), pyridine, (0.12 ml, 1.5 mmol) and copper(II) acetate (0.14 g, 0.75 mmol) in 1-methyl-2-pyrrolidinone (10 ml) was stirred at room temperature overnight. The mixture was filtered and the filtrate was treated with water (50 ml) and acidified with 1M hydrochloric acid to pH 6. The precipitate was removed by filtration and the product was recovered from the filtrate by extraction with ethyl acetate (3×20 ml). The combined organic extracts were washed with brine, dried over sodium sulfate, and the solvent removed under reduced pressure giving 0.07 g (50%) of the title compound: mp 258° C.; ¹H-NMR (DMSO-d₆): δ 0.67 (m, 4H), 1.33 (s, 3H), 7.11 (br s, 1H), 7.89 (br s, 1H), 11.25 (br s, 1H).

### EXAMPLE 5

6-Chloro-3-ethylamino-4H-thieno[3,2-e]-1,2,4-thiadiazine 1,1-dioxide

a) N-(5-Chloro-2-thienylsulfonyl)-N′-ethylquanidine

A solution of 5-chloro-2-thiophenesulfonyl chloride (10 g, 46 mmol) in ethyl ether (100 ml) was added to a stirred solution of N-ethylguanidine hydrochloride (5.7 g, 46 mmol) in 1N sodium hydroxide (100 ml), and the mixture was stirred at room temperature for 1 h. The resulting precipitate was isolated by filtration, washed with ethyl ether and dried to give 9.15 g (73%) of the title compound as a white solid: ¹H-NMR (DMSO-d₆): δ 1.02 (t, 3H), 3.09 (quint, 2H), 7.12 (d, 1H), 7.37 (d, 1H), 6.5-8.0 (broad peaks, 3H); LC-MS: m/z268/270 (M+1)⁺.

b) N-(5-Chloro-3-(dihydroxyboryl)-2-thienylsulfonyl)-N′-ethylquanidine

To a stirred solution of N-(5-chloro-2-thienylsulfonyl)-N′-ethylguanidine (4.0 g, 15 mmol) in dry tetrahydrofuran (50 ml) was added n-butyllithium (30 ml of a 2.5 M solution in hexane; 75 mmol) at −60° C. under nitrogen. After stirring at −60° C. for 1 h, the mixture was cooled to −70° C. and trimethyl borate (10.2 ml, 90 mmol) was. The mixture was stirred at −60° C. for 2 h, and then allowed to reach room temperature. Water (50 ml) and ethyl acetate (50 ml) was added to the mixture and pH was adjusted to 3-4 with 1M hydrochloric acid. The two phases were separated, the aqueous phase was extracted with ethyl acetate (2×25 ml), and the combined organic phases were washed with water, dried over sodium sulfate and evaporated to dryness. The residue was taken up in a mixture of water (100 ml) and ethyl acetate (50 ml), acidified to pH 2 with 1M hydrochloric acid, and stirred for 2 h. The two phases were separated, the aqueous phase was extracted with ethyl acetate (2×25 ml), and the combined organic phases were washed with water, dried over sodium sulfate and evaporated to dryness to give 5.3 g of the crude impure title compound as a brown oil, which was used in the next step without further purification.

c) 6-Chloro-3-ethylamino-4H-thieno[3,2-e]-1,2,4-thiadiazine 1,1-dioxide

A mixture of the crude N-(5-chloro-3-(dihydroxyboryl)-2-thienylsulfonyl)-N′-ethylguanidine(2 g), pyridine (1.03 ml, 12.8 mmol), 4A molecular sieves (2.8 g) and copper(II) acetate (0.21 g, 1.06 mmol) in 1-methyl-2-pyrrolidinone (80 ml) was stirred at room temperature for 2 h. The reaction mixture was filtered and the filtrate was poured into water (125 ml), acidified to pH 2 with 1M hydrochloric acid, and extracted with ethyl acetate (2×50 ml). The organic phase was dried over sodium sulfate and evaporated to dryness to give 3.2 g of the crude product. Purification by column chromatography on silica (ethyl acetate/petroleum ether 7:3) afforded a pure sample of the title compound: mp 271-274° C. (ethyl acetate); ¹H-NMR (DMSO-d₆): δ 1.11 (s, 3H), 3.21 (m, 2H), 7.03 (s, 1H), 7.29 (br, 1H), 11.0 (br s, 1H).

### EXAMPLE6

6-Chloro-3-octylamino-4H-thieno[3,2-e]-1,2,4-thiadiazine 1,1-dioxide

a) N-(5-Chloro-2-thienylsulfonyl)-N′-octylguanidine

A solution of 5-chloro-2-thiophenesulfonyl chloride (4.9 g, 22.5 mmol) in ethyl ether (50 ml) was added to a stirred suspension of N-octylguanidine hemisulfate (5.0 g, 22.7 mmol) in 1N sodium hydroxide (50 ml), and the mixture was stirred at room temperature for 90 min. The organic phase was separated, dried over sodium sulfate, concentrated, and triturated with heptane (25 ml) to afford 4.0 g (51%) of the title compound as an off-white solid: ¹H-NMR (DMSO-d₆): δ 0.86 (distorted t, 3H), 1.1-1.5 (m, 12H), 3.05 (t, 2H), 6.6-7.4 (very broad peak, 3H), 7.11 (d, 1H), 7.34 (d, 1H); LC-MS: m/z 352/354 (M+1)⁺.

b) N-(5-Chloro-3-(dihydroxyboryl)-2-thienylsulfonyl)-N′-octylguanidine

To a stirred solution of N-(5-chloro-2-thienylsulfonyl)-N′-octylguanidine (3.9 g, 11.1 mmol) in dry tetrahydrofuran (50 ml) was added n-butyllithium (22.2 ml of a 2.5 M solution in hexane, 55.4 mmol) at −60° C. under nitrogen. After stirring at −60° C. for 1 h, the mixture was cooled to −70° C. and trimethyl borate (7.55 ml, 66.5 mmol) was added. The mixture was stirred at −60° C. for 2½ h, and then allowed to reach room temperature. Water (50 ml) and ethyl acetate (50 ml) was added to the mixture and pH was adjusted to 2-3 with 1M hydrochloric acid. The two phases were separated, the aqueous phase was extracted with ethyl acetate (2×25 ml), and the combined organic phases were dried over sodium sulfate and evaporated to dryness. The residue was taken up in a mixture of water (100 ml) and ethyl acetate (50 ml), acidified to pH 2 with 1M hydrochloric acid, and stirred for 2 h. The two phases were separated, the aqueous phase was extracted with ethyl acetate (2×25 ml), and the combined organic phases were dried over sodium sulfate and evaporated to dryness to give 4.8 g of a semi solid product. Trituration with petroleum ether (100 ml) and drying in vacuo afforded 3.2 g of the crude title compound, which was used in the next step without further purification.

c) 6-Chloro-3-octylamino-4H-thieno[3,2-e]-1,2,4-thiadiazine 1,1-dioxide

A mixture of the crude N-(5-chloro-3-(dihydroxyboryl)-2-thienylsulfonyl)-N′-octylguanidine(3.2 g), pyridine (0.88 ml, 11.2 mmol), powdered 4 Å molecular sieves (4.3 g) and copper(II) acetate (1.0 g, 5.6 mmol) in dichloromethane (100 ml) was stirred at room temperature overnight. The reaction mixture was filtered and the filtrate was poured into water (150 ml), acidified to pH 2 with 1M hydrochloric acid, and the phases were separated. The aqueous phase was extracted with dichloromethane (2×50 ml) and the combined organic phases were dried over sodium sulfate and evaporated to dryness to give 3.1 g of the crude product as a brown oil.

Purification by column chromatography on silica (ethyl acetate/petroleum ether 7:3) afforded a pure sample of the title compound: mp 202-205° C.; ¹H-NMR (DMSO-d₆): δ 0.85 (t, 3H), 1.28 (m, 10H), 1.50 (m, 2H), 3.18 (q, 2H), 7.03 (s, 1H), 7.25 (br s, 1H), 10.2 (br s, 1H).

What is claimed is:

1. A process for the preparation of a compound of formula (I) 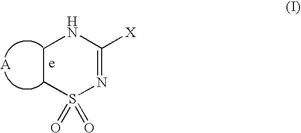
wherein
X is NR²R³, SR¹, S(═O)R¹, S(═O)₂R¹ or OR¹;
R¹ is hydrogen, C₃₋₆-cycloalkyl or (C₃₋₆-cycloalkyl)C₁₋₆-alkyl the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms, optionally being mono- or polysubstituted with halogen, cyano, trifluoromethyl, C₁₋₆-alkyl, C₁₋₆-alkoxy, C₁₋₆-alkoxy-C₁₋₆-alkyl, aryl, arylalkyl, hydroxy, oxo, nitro, amino, C₁₋₆-monoalkyl or dialkylamino; or straight or branched C₁₋₁₈-alkyl, C₂₋₁₈-alkenyl or C₂₋₁₈-alkynyl, each of the groups being optionally mono- or polysubstituted with halogen, hydroxy, C₁₋₆-alkoxy, C₁₋₆-alkylthio, C₃₋₆-cycloalkyl, nitro, amino, C₁₋₆- monoalkyl- or dialkylamino, cyano, oxo, formyl, acyl, carboxy, C₁₋₆-alkoxycarbonyl, carbamoyl, formylamino, C₁₋₆-alkylcarbonylamino, aryl, aryloxy, arylalkoxy; bicycloalkyl, aryl, heteroaryl, arylalkyl or heteroarylalkyl, each of the groups being optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;
R² is hydrogen; hydroxy; C₁₋₆-alkoxy; or C₁₋₆-alkyl, C₃₋₆-cycloalkyl, C₂₋₆- alkenyl or C₂₋₆-alkynyl optionally mono- or polysubstituted with halogen;
R³ is hydrogen, C₃₋₆-cycloalkyl or (C₃₋₆-cycloalkyl)C₁₋₆-alkyl, the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl; or
R³ is —OR⁴; —C(═Z)R⁴; —NR⁴R⁵; bicycloalkyl, aryl, heteroaryl, arylalkyl or heteroarylalkyl optionally mono- or polysubstituted with halogen, hydroxy, C₁₋₆-alkyl, C₁₋₆-alkoxy, aryloxy, arylalkoxy, nitro, amino, C₁₋₆-monoalkyl- or dialkylamino, cyano, oxo, acyl or C₁₋₆-alkoxycarbonyl;
R⁴ is hydrogen; C₃₋₆-cycloalkyl or (C₃₋₆-cycloalkyl)C₁₋₆-alkyl, the C₃₋₆-cycloalkyl group optionally being mono- or polysubstituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or polysubstituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;
Z is O or S;
R⁵ is hydrogen; C₁₋₆-alkyl; C₂₋₆-alkenyl; C₃₋₆-cycloalkyl optionally mono- or polysubstituted with C₁₋₆-alkyl, halogen, hydroxy or C₁₋₆-alkoxy; or
when R³ is —NR⁴R⁵, R⁴ and R⁵ together with the nitrogen atom form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or
when X is —NR²R³, R² and R³ together with the nitrogen atom may form a 3-12 membered mono- or bicyclic system, in which one or more of the carbon atoms may be exchanged with nitrogen, oxygen or sulfur, each of these ring systems optionally being mono- or polysubstituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;
A together with the carbon atoms forming bond e of formula I represents a 5 membered heterocyclic system comprising one or more nitrogen-, oxygen- or sulfur atoms, the heterocyclic system optionally being mono- or polysubstituted with halogen; C₁₋₁₈-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, aryl, arylalkyl, aryloxy, the aryl group optionally being mono- or polysubstituted with C₁₋₆-alkyl, perhalomethyl, halogen, hydroxy or C₁₋₆-alkoxy; C₁₋₆-alkoxycarbonyl; C₁₋₆-alkoxycarbonyl-C₁₋₆-alkyl; carbamyl; carbamylmethyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thiocarbamyl; thioureido; C₁₋₆-monoalkyl- or dialkylaminothiocarbonyl- amino; C₁₋₆-monoalkyl- or dialkylaminosulfonyl; carboxy; carboxy-C₁₋₆-alkyl; acyl; formyl; or a 5-6 membered nitrogen, oxygen or sulfur containing ring, optionally substituted with C₁₋₆-alkyl or phenyl, the phenyl group optionally being mono- or polysubstituted with C₁₋₆-alkyl, perhalomethyl, halogen, hydroxy or C₁₋₆-alkoxy; or
a salt thereof with a pharmaceutically acceptable acid or base, or an optical isomer thereof, or a tautomeric form thereof, or metabolites or prodrugs thereof,
comprising one of the following methods:
a) reacting a compound of formula (II) 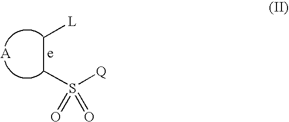
wherein A is as defined above, L is a leaving group selected from hydrogen, halogen or trimethylsilyl, and Q is halogen, with a compound of formula (III), 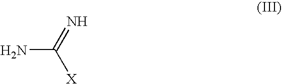
wherein X is NR²R³, wherein R² and R³ are defined above, or a suitable salt thereof, in the presence of a suitable base, to form a compound of formula (IV), 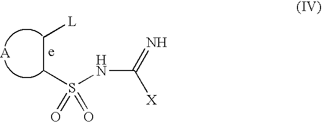
wherein A and L are as defined above and X is NR²R³; then reacting the compound of formula (IV) with B(OR′)(OR″)(Y), wherein Y is hydrogen or OR′″, R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V), 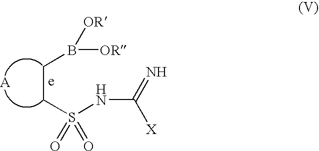
wherein A, R′ and R″ are as defined above and X is NR²R³, and thereupon cyclization of the compound of formula (V), optionally in the presence of a metal specie, to form the compound of formula (I); or
b) reacting a compound of formula (II) 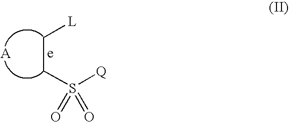
wherein A is as defined above, L is a leaving group selected from hydrogen, halogen or trimethylsilyl, and O is halogen, with a compound of formula (III), 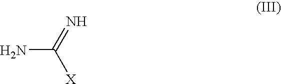
wherein X is SR¹, S(═O)R¹ or S(═O)₂R¹, wherein R¹ is defined above, or a suitable salt thereof, in the presence of a suitable base to form a compound of formula (IV), 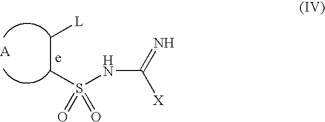
wherein A and L are as defined above and X is SR¹, S(═O)R¹ or S(═O)₂R¹, then reacting the compound of formula. (IV) with B(OR′)(OR″)(Y), wherein Y is hydrogen or OR′″, R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V), 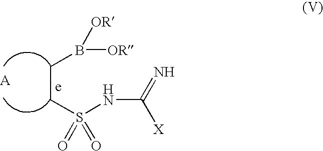
wherein A, R′ and R″ are as defined above and X is SR¹, S(═O)R¹ or S(═O)₂R¹, and thereupon cyclization of the compound of formula (V), optionally in the presence of a metal specie, to form the compound of formula (I); or
c) reacting a compound of formula (II) 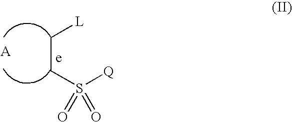
wherein A is as defined above, L is a leaving group selected from hydrogen, halogen or trimethylsilyl, and Q is halogen, with a compound of formula (III), 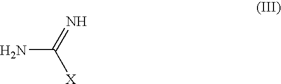
wherein X is OR¹, wherein R¹ is defined above, or a suitable salt thereof, in the presence of a suitable base, to form a compound of formula (IV) 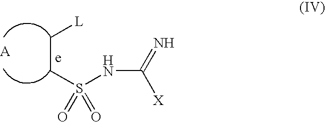
wherein A and L are as defined above and X is OR¹, then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V), 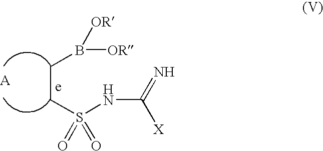
wherein A, R′ and R″ are as defined above and X is OR¹, and thereupon cyclization of the compound of formula (V), optionally in the presence of a metal specie, to form the compound of formula (I).

2. A process according to claim 1 comprising following method:
a) reacting a compound of formula (II) 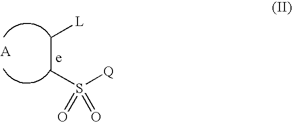
wherein A is as defined above, L is a leaving group selected from hydrogen, halogen or trimethylsilyl, and Q is halogen, with a compound of formula (III), 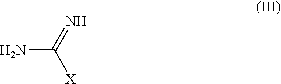
wherein X is NR²R³, wherein R² and R³ are defined above, or a suitable salt thereof, in the presence of a suitable base, to form a compound of formula (IV), 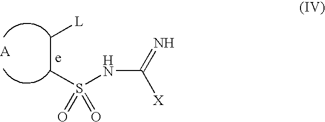
wherein A and L are as defined above and X is NR²R³; then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V), 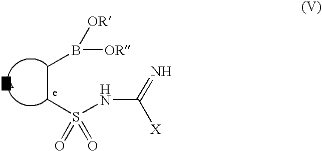
wherein A, R′ and R″ are as defined above and X is NR²R³, and thereupon cyclization of the compound of formula (V), optionally in the presence of a metal specie, to form the compound of formula (I).

3. A process according to claim 1 comprising following method:
b) reacting a compound of formula (II) 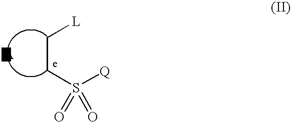
wherein A is as defined above, L is a leaving group selected from hydrogen, halogen or trimethylsilyl, and Q is halogen, with a compound of formula (III), 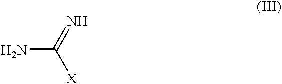
wherein X is SR¹, S(═O)R¹ or S(═O)₂R¹, wherein R¹ is defined above, or a suitable salt thereof, in the presence of a suitable base to form a compound of formula (IV), 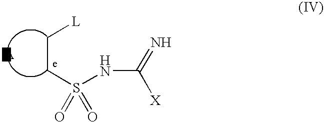
wherein A and L are as defined above and X is SR¹, S(═O)R¹ or S(═O)₂R¹, then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V), 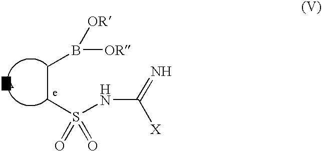
wherein A, R′ and R″ are as defined above and X is SR¹, S(═O)R¹ or S(═O)₂R¹, and thereupon cyclization of the compound of formula (V), optionally in the presence of a metal specie, to form the compound of formula (I).

4. A process according to claim 1 comprising following method:
c) reacting a compound of formula (II) 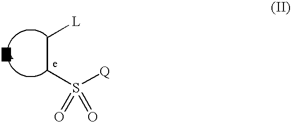
wherein A is as defined above, L is a leaving group selected from hydrogen, halogen or trimethylsilyl, and Q is halogen, with a compound of formula (III), 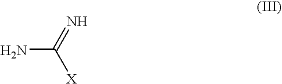
wherein X is OR′, wherein R¹ is defined above, or a suitable salt thereof, in the presence of a suitable base, to form a compound of formula (IV) 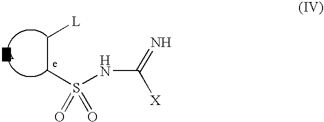
wherein A and L are as defined above and X is OR¹, then reacting the compound of formula (IV) with B(OR′)(OR″)(Y), wherein Y is hydrogen or OR′″; R′, R″ and R′″ independently are hydrogen or C₁₋₈-alkyl, or wherein OR′ and OR″ together with the boron atom either form or can be transformed into a 4-12 membered mono- or bicyclic system, to form a compound of formula (V), 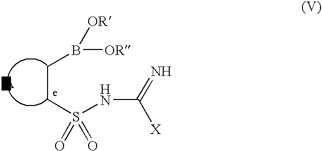
wherein A, R′ and R″ are as defined above and X is OR¹, and thereupon cyclization of the compound of formula (V), optionally in the presence of a metal specie, to form the compound of formula (I).

5. A process according to claim 1 wherein R² is hydrogen or C₁₋₆-alkyl, and R³ is hydrogen, C₃₋₆-cycloalkyl, (C₃₋₆-cycloalkyl)C₁₋₆-alkyl or straight or branched C₁₋₁₈-alkyl.

6. A process according to claim 2 wherein R² is hydrogen or C₁₋₆-alkyl, and R³ is hydrogen, C₃₋₆-cycloalkyl, (C₃₋₆-cycloalkyl)C₁₋₆-alkyl or straight or branched C₁₋₁₈-alkyl.

7. A process according to claim 1 wherein A together with the carbon atoms forming bond e of formula (I) represents a 5 membered heterocyclic system comprising one sulfur atom, the heterocyclic system optionally being substituted with halogen.

8. A process according to claim 1 wherein R′ and R″ are hydrogen.

9. A process according to claim 1 wherein OR′ and OR″ together with the boron atom form a 1,3,2-dioxaborolan-2-yl, 4,4,5,5-tetramethyl-1,3,2-dioxaborolan-2-yl, 1,3,2-dioxaborinan-2-yl, 5,5-dimethyl-1,3,2-dioxaborinan-2-yl or a 1,3,2-benzodioxaborol-2-yl group.

10. A process according to claim 1 wherein the compounds of formula (I) is selected from the group consisting of:
3-Amino-6-chloro-4H-thieno[3,2-e]-1,2,4-thiadiazine 1,1-dioxide,
6-Chloro-3-isopropylamino-4H-thieno[3,2-e]-1,2,4-thiadiazine 1,1-dioxide,
6-Chloro-3-(1-methylcyclopropyl)amino-4H-thieno[3,2-e]-1,2,4-thiadiazine 1,1-dioxide,
6-Chloro-3-ethylamino-4H-thieno[3,2-e]-1,2,4-thiadiazine 1,1-dioxide, and
6-Chloro-3-octylamino-4H-thieno[3,2-e]-1,2,4-thiadiazine 1,1-dioxide, or
a salt thereof with a pharmaceutically acceptable acid or base, or an optical isomer thereof, or a tautomeric form thereof, or metabolites or prodrugs thereof.

11. A process according to claim 1 wherein the bases are selected from sodium hydroxide, potassium carbonate, cesium carbonate, potassium hydroxide, pyridine, triethylamine, butyl lithium, hexyl lithium, isopropyl magnesium chloride.

12. A process according to claim 1 wherein the metal specie is selected from copper bronze, copper oxide, copper chloride, copper bromide, copper iodide, copper acetate.
